(19) United States
(12) Patent Application Publication
(10) Pub. No.: US 2023/0226211 A1
(43) Pub. Date: Jul. 20, 2023

(54) CD25-TARGETED IL-2 FOR INCREASING CD4 T CELL FORMATION AND TREATMENT OF INFECTIONS

(71) Applicant: UNIVERSITY OF CENTRAL FLORIDA RESEARCH FOUNDATION, INC., Orlando, FL (US)

(72) Inventors: Tara M. STRUTT, Orlando, FL (US); Karl Kai MCKINSTRY, Orlando, FL (US)

(21) Appl. No.: 17/920,942

(22) PCT Filed: Apr. 26, 2021
(86) PCT No.: PCT/US2021/029077

§ 371 Date: Oct. 24, 2022

Related U.S. Application Data
(60) Provisional application No. 63/015,449, filed on Apr. 24, 2020.

(51) Int. Cl. A61K 47/68; A61P 31/16

(52) U.S. Cl. CPC A61K 47/6845; A61K 47/6813; A61P 31/16

(57) ABSTRACT

Disclosed are compositions comprising an IL-2:anti-IL-2 antibody (Ab) complex (IL-2C) and methods of using said N compositions for the treatment of microbial infections, autoimmune diseases, autoinflammatory diseases, or cancers as well the treatment of inflammatory conditions or reduction in inflammation caused by said microbial infections, autoimmune diseases, autoinflammatory diseases, or cancers.

This Application claims the benefit of U.S. Provisional Application No. 63/015,449, filed on Apr. 24, 2020 which is incorporated herein by reference in its entirety.

This invention was made with government support under Grant No. R21AI117457 and R21HD093948 awarded by the National Institutes of Health. The government has certain rights in the invention.

### I. BACKGROUND

Influenza A virus (IAV) remains a significant public health concern despite widespread vaccination efforts. Certain individuals are highly susceptible to infection and suffer from serious disease requiring hospitalization. Serious influenza can be fatal and is often associated with the development of an acute respiratory distress syndrome (ARDS) characterized by an uncontrolled inflammatory ‘cytokine storm’ and severely compromised lung function. Current treatments for severe IAV and ARDS are limited to the use of high doses of antivirals, such as Oseltamivir phosphate (tradename Tamiflu) that is most effective when given early during infection, and invasive ventilation, which can double the risk of death. Innovative clinical interventions able to prevent severe respiratory virus infection are urgently needed, especially approaches that can be initiated at later stages of infection.

### II. SUMMARY

Disclosed are IL-2:anti-IL-2 antibody (Ab) complex (IL-2C) and methods of using said compositions for the treatment of microbial infections, autoimmune diseases, autoinflammatory diseases, or cancers as well the treatment of inflammatory conditions or reduction in inflammation caused by said microbial infections, autoimmune diseases, autoinflammatory diseases, or cancers.

In one aspect, disclosed herein are compositions comprising an IL-2:anti-IL-2 antibody (Ab) complex (IL-2C), wherein the anti-IL-2 antibody (such as for example the human anti-IL-2 antibody clone F5111.2 or its mouse equivalent JES6-1A12) binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

Also disclosed herein are methods of treating, decreasing, inhibiting, reducing, ameliorating, and/or preventing a microbial infection, autoimmune disease, autoinflammatory disease, or cancer in a subject comprising administering to the subject the composition of any preceding aspect. For example, disclosed herein are methods treating, decreasing, inhibiting, reducing, ameliorating, and/or preventing a microbial infection, autoimmune disease, autoinflammatory disease, or cancer in a subject comprising administering to the subject an IL-2:anti-IL-2 antibody (Ab) complex (IL-2C), wherein the anti-IL-2 antibody (such as for example the human anti-IL-2 antibody clone F5111.2 or its mouse equivalent JES6-1A12) binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor. In one aspect, the composition is administered can be administered 1, 2, 3, 4, 5 6, 7, 8, 9, 10, 11, 12, 13, 14, 15, 16, 17, 18, 19, 20, 21, 22, 23, 24, 25, 26, 27, 28, 29, 30, 31, 45, 58, 59, 60, 61, 75, 90 days, 4, 5, 6, 7, 8, 9, 10, 11, or 12 months after onset of the microbial infection (such as, for example, 1, 2, 3, or 4 days post infection), autoimmune disease, autoinflammatory disease, or cancer.

In one aspect, disclosed herein are methods of treating, decreasing, inhibiting, reducing, ameliorating, and/or preventing a microbial infection, autoimmune disease, autoinflammatory disease, or cancer of any preceding aspect, wherein the microbial infection is a viral infection, and wherein the viral infection is an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are methods of treating, decreasing, inhibiting, reducing, ameliorating, and/or preventing a microbial infection, autoimmune disease, autoinflammatory disease, or cancer of any preceding aspect, wherein the microbial infection is a bacterial infection, and wherein the bacterial infection is an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Haemophilus species, Clostridium tetani, other Clostridium species, Yersinia enterolitica, and other Yersinia species.

In one aspect, disclosed herein are methods of treating, decreasing, inhibiting, reducing, ameliorating, and/or preventing a microbial infection, autoimmune disease, autoinflammatory disease, or cancer of any preceding aspect, wherein the microbial infection is a fungal infection, and wherein the fungal infection is an infection with a fungus selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis camii, Penicillium marneffi, and Alternaria alternata.

Also disclosed herein are methods of treating, decreasing, inhibiting, reducing, ameliorating, and/or preventing a microbial infection, autoimmune disease, autoinflammatory disease, or cancer of any preceding aspect, wherein the microbial infection is a parasitic infection, and wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In one aspect, disclosed herein are methods of treating, decreasing, inhibiting, reducing, ameliorating, and/or preventing a microbial infection, autoimmune disease, autoinflammatory disease, or cancer of any preceding aspect, further comprising administering to the subject an anti-microbial agent.

Also disclosed herein are methods of treating, decreasing, inhibiting, reducing, ameliorating, and/or preventing a microbial infection, autoimmune disease, autoinflammatory disease, or cancer of any preceding aspect, further comprising administering to the subject an anti-cancer agent.

In one aspect, disclosed herein are methods of treating, decreasing, inhibiting, reducing, ameliorating, and/or preventing an inflammatory condition (such as, for example, acute inflammation, acute respiratory distress syndrome, subacute inflammation, chronic inflammation, organ-specific inflammation, systemic inflammation, or sepsis) or treating, decreasing, inhibiting, reducing, ameliorating, and/or preventing inflammation caused by a microbial infection, autoimmune disease, autoinflammatory disease, or cancer in a subject comprising administering to the subject the composition of any preceding aspect. For example, disclosed herein are methods of treating, decreasing, inhibiting, reducing, ameliorating, and/or preventing an inflammatory condition (such as, for example, acute inflammation, acute respiratory distress syndrome, subacute inflammation, chronic inflammation, organ-specific inflammation, systemic inflammation, or sepsis) or treating, decreasing, inhibiting, reducing, ameliorating, and/or preventing inflammation caused by a microbial infection, autoimmune disease, autoinflammatory disease, or cancer in a subject comprising administering to the subject an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor. In one aspect, the composition is administered can be administered 1, 2, 3, 4, 5 6, 7, 8, 9, 10, 11, 12, 13, 14, 15, 16, 17, 18, 19, 20, 21, 22, 23, 24, 25, 26, 27, 28, 29, 30, 31, 45, 58, 59, 60, 61, 75, 90 days, 4, 5, 6, 7, 8, 9, 10, 11, or 12 months after onset of the microbial infection (such as, for example, 1, 2, 3, or 4 days post infection), autoimmune disease, autoinflammatory disease, or cancer.

Also disclosed herein are methods of enhancing or increasing an immune response (including but not limited to responses from T cells (such as, for example, and increase in CD8 T cells (including, but not limited to, effector, central memory, effector memory, and peripheral memory CD8 T cells), CD4 T cells (including, but not limited to T_{H}1, T_{H}2, and T_{H}17 CD4 T cells), B cell, regulatory CD4 T cells (Tregs), NK cells, NK T cells, γδ T cells, innate lymphoid cells (including, but not limited to ILC1, ILC2, and ILC3)) and/or enhancing or increasing the formation of immunological memory (including, but not limited to memory CD8 T cells (including, but not limited to, central memory, effector memory, and peripheral memory CD8 T cells), memory CD4 T cells (including, but not limited to T_{H}1, T_{H}2, and T_{H}17 CD4 T cells), memory B cells, plasma cells, memory NK cells, and memory NK T cells), to a microbial infection, autoimmune disease, autoinflammatory disease, or cancer in a subject comprising administering to the subject the composition of any preceding aspect. For example, disclosed herein are methods of enhancing or increasing a T cell response to a microbial infection, autoimmune disease, autoinflammatory disease, or cancer in a subject comprising administering to the subject an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor. In one aspect, the composition is administered can be administered 1, 2, 3, 4, 5 6, 7, 8, 9, 10, 11, 12, 13, 14, 15 days after onset of the microbial infection or cancer.

In one aspect, disclosed herein are methods of any preceding aspect further comprising the administration of additional inflammatory modulating elements including, but not limited to anti-CD70, anti-CD28, depletion of NK cells, and/or PD-1 agonists (such as, for example ANB030), CTLA-4 agonist (such as, for example abatacept and belatacept), TIM-3 agonist, and/or LAG-3 agonists.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, and 1E show that JES6 IL-2C treatment increases diverse lymphocyte subsets. Mice were treated for 3 days with JES6 IL-2C and on the fourth day spleens were analyzed by flow cytometry. FIG. 1A shows the total number of regulatory CD4 T cells (CD25⁺FOXP3⁺) and (1B) representative staining from untreated and treated mice of CD25 and FOXP3 co-staining. FIG. 1C shows CD25 mean fluorescence intensity on FOXP3+CD4 T cells and (1D) frequency of Ki-67⁺FOXP3⁺ cells with representative staining (dark shaded histograms are total CD4 T cells). FIG. 1E shows total numbers of CD4 T cells, CD8 T cells, NK cells, ILCs, and γδ T cells in mice treated only with PBS (white, n=6) or with JES6-IL-2C (grey, n=4). Representative results from 1 of 3 replicate experiments and *P<0.05, ***P<0.001, ****P<0.0001 following Students t-test analysis.

FIGS. 2A, 2B, 2C, and 2D show that JES6-IL-2C treatment increases diverse lymphocyte subsets. Mice were treated for 3 days with JES6-IL-2C and on the fourth day spleens and lungs from 4 to 6 mice per group were analyzed by flow cytometry. Representative frequencies and gating strategies employed to enumerate (2A) CD45⁺CD90⁺, CD4⁺, CD8⁺, and (2B) CD45⁺CD90⁺γδ TcR⁺ lymphocytes and their expression of CD4 and CD8. FIG. 2C shows CD45⁺, CD49b⁺, CD3⁻ NK lymphocytes and gated NK cell expression of CD11b (black line), (2D) CD45⁺, Lineage (Lin)⁻, CD3⁺, CD90.1⁺ innate lymphoid cells (ILC) and gated ILC expression of CD127. Shaded histograms in b and c are appropriate staining controls

FIGS. 3A and 3B show that JES6 IL-2C treatment induces wide-spread systemic expression of inflammatory cytokines and chemokines. Mice were treated with JES6 IL-2C or with PBS alone for 3 consecutive days. On the fourth day, serum was harvested and was analyzed by Luminex for protein levels of (3A) inflammatory cytokines and chemokines typically associated with Th1 responses and (3B) cytokines associated with Th2 responses (n=4 mice per group). Results from 1 of 3 replicate experiments *P<0.05, **P<0.01, ***P<0.001, ****P<0.0001 following Students t-test analysis.

FIGS. 4A, 4B, 4C, 4D, and 4E show that systemic JES6 IL-2C treatment induces inflammation in the lung. Mice treated systemically with IL-2C or PBS alone were analyzed for changes in lymphocyte populations and inflammation in the lungs. Shown in (4A) the number of regulatory T cells with (4B) representative staining from treated and untreated mice. FIG. 4C shows total numbers of CD4 T cells, CD8 T cells, NK cells, γδ T cells, and ILC (n=4 mice per group). Lung homogenates from separate groups of JES6 IL-2C treated or control mice (n=4 per group) were analyzed for protein levels of inflammatory factors associated with (4D) Th1 or (4E) Th2 responses. Results from one of 3 replicate experiments and *P<0.05, **P<0.01, ***P<0.001, following Students t-test analysis.

FIGS. 5A and 5B show that JES6-IL-2C-driven lung inflammatory responses are CD25 dependent. Groups of 4 mice were treated i.p. with JES6-IL-2C containing stated amounts of recombinant murine IL-2 or with PBS alone (0 μg). One group of mice receiving IL-2C containing 2 μg of IL-2 was pre-treated the day before initiation of IL-2C administration with 500 μg CD25-blocking antibody (clone PC-61.5.3). Results from one of two similar experiments.

FIGS. 6A and 6B show that intranasally administered JES6-IL-2C drives potent inflammatory responses in the lung. Groups of 4 mice were treated intranasally with either 50 L of PBS alone or 50 μL of JES6-IL-2C for three consecutive days. On the fourth day, lung homogenates and serum were harvested and protein levels of the stated cytokines and chemokines assessed by Luminex. Results from one of two similar experiments.

FIGS. 7A, 7B, 7C, 7D, and 7E show that JES6 IL-2C treatment improves outcomes of IAV infection. Groups of mice were infected with a sublethal 0.2 LD₅₀ dose of IAV and treated i.p. with either PBS alone or with JES6 IL-2C. On day 4, levels of stated cytokines and chemokines detected by Luminex from lung homogenates and associated with either (7A) Th1 or (7B) Th2 responses from 4 mice per group were determined. The average level of analytes detected following JES6 IL-2C administration alone is depicted as a dashed line in each graph. Separate mice were infected with IAV and treated with either PBS alone (white circle) or with JES6 IL-2C for 3 days. (7C) Shown is the survival summarizing 4 mice per group. Mice treated as in (7C) were harvested at 7 dpi and assessed for histopathological changes. Shown in (7D) is the cumulative histopathology score broken down by alveolar inflammation (AV, black), bronchial inflammation (BR, grey), and perivascular inflammation (PV, white). Groups of 5 mice were treated with either PBS or JES6 IL-2C i.p. for 3 consecutive days and (7E) were assessed on stated days for respiratory rate (RR in breaths per min), minute volume (MV in cm³ per min), and PenH (Enhanced pause). All results representative of at least 2 replicate experiments and *P<0.05, **P<0.01, ***P<0.001, ****P<0.0001 following Students t-test (7A and 7B) or one-way ANOVA analysis (7D).

FIG. 8 shows that JES6-IL-2C treatment suppresses bronchial histopathology during IAV infection. Uninfected and sublethal 0.2LD₅₀ A/PR8-OVA_{II} infected BALB/c mice were treated with PBS or JES6-IL-2Cs containing 2 μg of IL-2 for 3 days. Representative photomicrographs of H & E stained tissue sections of lungs on 4 dpi are shown, Br: bronchus; Ar: artery.

FIGS. 9A, 9B, and 9C show distinct and overlapping patterns in IL-2C induced inflammation. Separate groups of uninfected or IAV infected mice were treated with PBS, JES6 IL-2C, or S4B6 IL-2C for 3 consecutive days. On the fourth day, lung homogenates were harvested and analyzed by Luminex for protein levels of inflammatory cytokines and chemokines. A heat map of the average amount of analytes significantly induced with IL-2C complex treatment in (9A) uninfected mice and (5B) sublethally 0.2 LD₅₀ IAV infected mice (3 mice per group; 1 of 3 experiments). FIG. 9C shows a Venn diagram depicting analytes significantly induced by both IL-2C or uniquely induced by JES6 IL-2C or S4B6 IL-2C during IAV infection.

FIGS. 10A, 10B, 10C, and 10D show JES6 IL-2C deliver pro-memory signals to CD4 T cells responding to IAV. BALB/c mice received congenically marked Il2^{-/-} DO11.10 CD4 T cells followed by priming with low-dose 0.2 LD₅₀ PR8-OVA_{II}. Groups of mice were either treated with S4B6 IL-2C (black line), JES6 IL-2C (grey line), or PBS alone (filled histogram) from 5-7 dpi. At 7 dpi, donor cells gated as in (10A) were analyzed for expression of CD25 and CD127, with representative staining and summary MFI analysis from 3 mice per group shown in (10B) and (10C), respectively. (10D) At 28 dpi, the total number of donor cells was enumerated in the spleen, dLN, and lung from groups of 3 to 4 mice treated with either IL-2C or with PBS alone. Summary of 2 replicate experiments and *P<0.05, **P<0.01 determined by one-way ANOVA analysis.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular IL-2:anti-IL-2 antibody complex (IL-2C) is disclosed and discussed and a number of modifications that can be made to a number of molecules including the IL-2C are discussed, specifically contemplated is each and every combination and permutation of IL-2C and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Interleukin-2 (IL-2) is a critical cytokine for orchestrating optimal immune responses. IL-2 acts as an autocrine T cell growth factor and can signal in a paracrine manner to promote the activation of other leukocyte subsets, most notably NK cells and CD8 T cells. However, IL-2 is also central to the maintenance and function of regulatory CD4 T cells (Tregs) that constrain immune responses and limit immunopathology. These divergent activities of IL-2 have been shown in diverse models and have been exploited clinically. Many strategies are being developed to specifically engage the pro-versus anti-inflammatory properties of IL-2 in context-dependent situations. For example, exogenously administered IL-2 can be targeted to either the a (CD25) or 3 (CD122) chain of the IL-2 receptor by using IL-2:anti-IL-2 antibody (Ab) complexes (IL-2C) made with different monoclonal Abs. In the mouse, the Ab clone S4B6 forms pro-inflammatory IL-2C that preferentially signal cells expressing high CD122, predominantly CD8 T and NK cells, while the anti-inflammatory IL-2C made with Ab clone JES6-1A12 (JES6) targets IL-2 to CD25 expressing cells, most notably Tregs in the steady state. The human IL-2 antibody F5111.2 targets IL-2 to CD25 in a manner similar to JES6.

In one aspect, disclosed herein are compositions comprising an IL-2:anti-IL-2 antibody (Ab) complex (IL-2C), wherein the anti-IL-2 antibody (such as for example the human anti-IL-2 antibody clone F5111.2 or its mouse equivalent JES6-1A12) binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution, and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal, or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids, and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex, and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Treating Microbial Infections and Controlling Inflammation

We show herein that IL-2 secreted by memory CD4 T cells responding to influenza A virus (IAV) can promote disease symptoms by increasing the production of inflammatory cytokines and chemokines in the lung. As part of these studies we treated naive mice or mice infected intranasally with a sublethal 0.2 LD₅₀ dose of the mouse-adapted IAV strain A/PuertoRico/8/1934 (A/PR8) for three days with S4B6 IL-2C and found that such treatment induced a remarkably broad inflammatory response that synergizes with IAV infection to exacerbate disease. We used this regime of IL-2C treatment as it delivers physiologically relevant IL-2 signals to IL-2 receptor-expressing CD4 T cells that promote memory formation during IAV infection, and similar protocols are widely employed in many different murine models.

How JES6 IL-2Cs that target CD25-expressing cells affect inflammatory cytokine and chemokine production systemically as well as in tissues such as the lung is not well-characterized. Here, we determine the impact of JES6 IL-2C on acute inflammation when given to naive mice and to mice challenged with IAV. We confirmed the treatment boosted T reg numbers and innate lymphoid cell populations (ILC) in the spleen as well as in the lung. However, JES6 IL-2C treatment drove an acute systemic inflammatory response defined by elevated levels of a diverse suite of cytokines and chemokines detected in serum and in lungs. JES6 IL-2C given to mice also challenged with low dose IAV enhanced levels of IFN-γ paradoxically at the same time as several Th2-associated factors, to levels above those detected in mice receiving either IAV or IL-2C alone. While treatment of IAV-infected mice with S4B6 IL-2C containing 2 μg of IL-2 results in acute death of all treated mice, IAV infected mice treated with JES6 IL-2C all survive infection. Furthermore, JES6 IL-2C treatment reduced the extent of lung immunopathology associated with IAV infection.

Given the differential outcome of JES6 versus S4B6 IL-2C treatment, we directly compared the inflammatory response induced by each in uninfected as well as in IAV infected mice. The results clearly show shared elements and unique patterns in the inflammatory milieu induced by JES6 IL-2C in the absence and presence of infection, demonstrating a complex governance of cytokine and chemokine expression, especially during IAV infection.

It is understood and herein contemplated that the effect that therapeutic effect observed relative influenza A infections is not limited to said virus or inflammation resulting from an influenza infection but can be extended to any microbial infection where the infection or disease state in is a result of IL-2 mediated inflammation. Accordingly, disclosed herein are methods of treating, decreasing, inhibiting, reducing, ameliorating, and/or preventing a microbial infection, autoimmune disease, autoinflammatory disease, or cancer in a subject comprising administering to the subject any of the IL-2C compositions disclosed herein (including, but not limited to a human IL-2 complexed with a human or humanized anti-IL-2 antibody (such as, for example, F5111.2).

In one aspect, disclosed herein are methods of treating, decreasing, inhibiting, reducing, ameliorating, and/or preventing a microbial infection, autoimmune disease, autoinflammatory disease, or cancer, wherein the microbial infection is a viral infection, and wherein the viral infection is an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are methods of treating, decreasing, inhibiting, reducing, ameliorating, and/or preventing a microbial infection, autoimmune disease, autoinflammatory disease, or cancer, wherein the microbial infection is a bacterial infection, and wherein the bacterial infection is an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Haemophilus species, Clostridium tetani, other Clostridium species, Yersinia enterolitica, and other Yersinia species.

In one aspect, disclosed herein are methods of treating, decreasing, inhibiting, reducing, ameliorating, and/or preventing a microbial infection, autoimmune disease, autoinflammatory disease, or cancer, wherein the microbial infection is a fungal infection, and wherein the fungal infection is an infection with a fungus selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

Also disclosed herein are methods of treating, decreasing, inhibiting, reducing, ameliorating, and/or preventing a microbial infection, autoimmune disease, autoinflammatory disease, or cancer, wherein the microbial infection is a parasitic infection, and wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

It is understood and herein contemplated that despite the ability of the disclosed compositions to inhibit microbial virulence and effectuate microbial clearance in tissue without the addition of an anti-microbial agent, there can be instances where the addition (either in the composition itself or as a separate administration) of an anti-microbial is desired. Accordingly, disclosed herein are methods of treating, decreasing, inhibiting, reducing, ameliorating, and/or preventing a microbial infection, autoimmune disease, autoinflammatory disease, or cancer, further comprising administering to the subject an anti-microbial agent. Anti-microbial agents can comprise any antibiotics, antibodies, small molecules, and functional nucleic acids (siRNA, RNAi, anti-sense oligonucleotides), that directly attack the infecting microbe or alter host conditions rendering the host system inhospitable to the microbe. Such agents include, but are not limited to Abacavir, Acyclovir, Adefovir, Amantadine, Amprenavir, Ampligen, Arbidol, Atazanavir, Atripla, Balavir, Beta-D-N4-hydroxycitidine (NHC, EIDD-1931), Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Hydroxy-chloroquine, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Nitazoxanide, Norvir, Oseltamivir, Peginterferon alfa-2a, Penciclovir, Peramivir, Pleconaril, Podophyllotoxin, Raltegravir, Remdecivir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, Clofazimine; Dapsone; Capreomycin; Cycloserine; Ethambutol (Bs); Ethionamide; Isoniazid; Pyrazinamide; Rifampicin; Rifabutin; Rifapentine; Streptomycin; Arsphenamine; Chloramphenicol (Bs); Fosfomycin; Fusidic acid; Metronidazole; Mupirocin; Platensimycin; Quinupristin/Dalfopristin; Thiamphenicol; Tigecycline (Bs); Tinidazole; Trimethoprim (Bs); aminoglycosides such as, for example, Amikacin, Gentamicin, Kanamycin, Meropenem, Neomycin, Netilmicin, Tobramycin, Paromomycin, Streptomycin, Spectinomycin, Nitazoxanide, Melarsoprol Eflomithine, Metronidazole, Tinidazole, Miltefosine, Mebendazole, Pyrantel pamoate, Thiabendazole, Diethylcarbamazine, Ivermectin, Niclosamide, Praziquantel, Albendazole, Praziquantel, Rifampin, Amphotericin B, Fumagillin, Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Posaconazole, Propiconazole, Ravuconazole, Terconazole, Voriconazole, Abafungin, Anidulafungin, Caspofungin, Micafungin, Aurones, Benzoic acid, Ciclopirox, Flucytosine, Griseofulvin, Haloprogin, Tolnaftate, Undecylenic acid, Crystal violet, Balsam of Peru, Orotomide, Miltefosine, ansamycins, such as, for example, geldanamycin, rifaximin, herbimycin; Carbapenems, such as, for example, Ertapenem, Doripenem, Imipenem/Cilastatin, and Meropenem; Cephalosporins, such as, for example, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole; Glycopeptides, such as, for example Teicoplanin, Vancomycin, Telavancin, Dalbavancin, and Oritavancin; Lincosamides (Bs), such as, for example, Clindamycin and Lincomycin; Lipopeptides, such as, for example, Daptomycin; Macrolides (Bs), such as, for example, Azithromycin, Clarithromycin, Erythromycin, Roxithromycin, Telithromycin, and Spiramycin; Monobactams, such as, for example, Aztreonam; Nitrofurans, such as, for example, Furazolidone and Nitrofurantoin (Bs); Oxazolidinones (Bs), such as, for example, Linezolid, Posizolid, Radezolid, and Torezolid; Penicillins, such as, for example, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; Polypeptides, such as, for example, Bacitracin, Colistin, and Polymyxin B; Quinolones/Fluoroquinolones, such as, for example, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; Sulfonamides (Bs), such as, for example, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), and Sulfonamidochrysoidine (archaic); Tetracyclines (Bs), such as, for example, Demeclocycline, Doxycycline, Metacycline, Minocycline, Oxytetracycline, and Tetracycline; monoclonal antibodies such as, for example, Actoxumab, Atidortoxumab, Berlimatoxumab, Bezlotoxumab, Cosfroviximab, Edobacomab, Felvizumab, Firivumab, Foravirumab, Larcaviximab, Motavizumab, Navivumab, Panobacumab, Palivizumab, Porgaviximab, CR6261, Rafivirumab, Pagibaximab, Obiltoxaximab, Ibalizumab, Regavirumab, Rmab, Sevirumab, Rivabazumab pegol, Tefibazumab, Suvratoxumab, and Tuvirumab; and checkpoint inhibitors; Pembrolizumab, Nivolumab, Atezolizumab, Avelumab, Durvalumab, pidilizumab, AMP-224, AMP-514, PDR001, cemiplimab, and Ipilimumab.

In response to infection with a microbe such as, for example, a virus, bacterium, fungus, or parasite, the host immune system attempts to eliminate the infecting microbe by employing arms of the innate and adaptive immune systems including the production of cytokines, antibodies, and effector mechanisms of granulocyte, monocyte, macrophage, dendritic cell, innate lymphoid cells, NK cells, NK T cells, T cells, B cells, and plasma cells. In any microbial inflammation, inflammatory signaling cascades, which are initiated by cell responses to microbial virulence factors and endogenous cytokines, culminate in the upregulatation inflammatory gene networks. Unchecked, this genomic reprogramming (genomic storm) leads to endothelial dysfunction, multi-organ failure and ultimately fatal shock, known as septic shock, that represents the ultimate end stage of microbial inflammation, one of the 10 leading causes of death in developed and developing countries.

“Microbial inflammation” refers to a condition associated with its cardinal signs such as redness, swelling, increase in temperature, pain, and impairment of organ function such as disordered respiration as a result of the epithelial injury with adjacent microvascular endothelial injury in the lungs (and other organs) due to a microbial infection such as a virus, bacteria, fungi, or parasite. That is, “Microbial inflammation” is a mechanism of disease caused by infection (“microbial insult”). Microbial inflammation evolves from innate immune response to an infection due to a microbe such as, for example, a virus, bacterium, fungus, or parasite. Thus, the microbial injury caused by microbial virulence factors is aggravated by the host-produced inflammatory mediators that impede the clearance of invading microbes and add insult to organ's injury. It is understood and herein contemplated that the microbial inflammation and its end stage, sepsis can result from any microbial insult elicited by known (or unknown) virulence factors and microbial antigens.

The innate and adaptive immune response to infecting pathogen (disease-causing microorganism) can include the burst in production of cytokines, chemokines, and proteolytic enzymes by granulocytes, monocytes, macrophages, dendritic cells, mast cells, innate lymphoid cells, T cells, B cells, NK cells, and NK T cells. Microbial inflammation can be localized to a specific organ- or can be systemic. Microbial inflammation can proceed in stages from acute to subacute and chronic with attendant tissue destruction and subsequent fibrosis. Left unchecked, the acute microbial inflammation can lead to sepsis and septic shock, the end stage of microbial inflammation. As the disclosed compositions have a profound effect on inflammation, many of the inflammatory complications can be controlled by administration of the IL-2C. Thus, in one aspect, disclosed herein are methods of treating, decreasing, inhibiting, reducing, ameliorating, and/or preventing an inflammatory condition (such as, for example, acute inflammation, acute respiratory distress syndrome, subacute inflammation, chronic inflammation, organ-specific inflammation, systemic inflammation, or sepsis) or treating, decreasing, inhibiting, reducing, ameliorating, and/or preventing inflammation caused by a microbial infection in a subject comprising administering to the subject the composition of any preceding aspect. For example, disclosed herein are methods of treating, decreasing, inhibiting, reducing, ameliorating, and/or preventing an inflammatory condition (such as, for example, acute inflammation, acute respiratory distress syndrome, subacute inflammation, chronic inflammation, organ-specific inflammation, systemic inflammation, or sepsis) or treating, decreasing, inhibiting, reducing, ameliorating, and/or preventing inflammation caused by a microbial infection, autoimmune disease, autoinflammatory disease, or cancer in a subject comprising administering to the subject an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

It is understood and herein contemplated that administration of a composition comprising an IL-2C alone may not be sufficient to address all the inflammatory responses in a subject. Thus, in one aspect, disclosed herein are methods of treating a microbial infection and/or an inflammatory condition or inflammation caused by a microbial infection further comprising the administration of additional inflammatory modulating elements including, but not limited to anti-CD70, anti-CD28, depletion of NK cells, and/or PD-1 agonists (such as, for example ANB030), CTLA-4 agonist (such as, for example abatacept and belatacept), TIM-3 agonist, and/or LAG-3 agonists).

The disclosed compositions modulate inflammatory responses and thus, can have a beneficial effect of reducing what could be a severe reaction to an infection to a less severe response. This effect can occur any time after infection. For example, administration can occur 1, 2, 3, 4, 5 6, 7, 8, 9, 10, 11, 12, 13, 14, 15, 16, 17, 18, 19, 20, 21, 22, 23, 24, 25, 26, 27, 28, 29, 30, 31, 45, 58, 59, 60, 61, 75, 90 days, 4, 5, 6, 7, 8, 9, 10, 11, or 12 months after onset of the microbial infection. In some instances of acute infections (for example, an influenza infection) administration can occur after infection but before the onset of severe inflammatory responses occurs (for example, 1, 2, 3, or 4 days post infection).

D. Methods of Treating Autoimmune Disease

As used herein, “autoimmune disease” refers to a set of diseases, disorders, or conditions resulting from an adaptive immune response (T cell and/or B cell response) against the host organism. In such conditions, either by way of mutation or other underlying cause, the host T cells and/or B cells and/or antibodies are no longer able to distinguish host cells from non-self-antigens and attack host cells bearing an antigen for which they are specific. Examples of autoimmune diseases include, but are not limited to graft versus host disease, transplant rejection, Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal pemphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)). In one aspect, disclosed herein are methods of treating, decreasing, inhibiting, reducing, ameliorating, and/or preventing an autoimmune diseases or inflammatory symptoms associated with an autoimmune comprising administering to the subject with an autoimmune disease a therapeutically effective amount of any of the IL-2C compositions disclosed herein (such as for example IL-2/F5111.2 antibody complex). For example, disclosed herein are methods of treating, decreasing, inhibiting, reducing, ameliorating, and/or preventing an autoimmune disease or methods of treating, decreasing, inhibiting, reducing, ameliorating, and/or preventing inflammation caused by an autoimmune disease in a subject comprising administering to the subject an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

The disclosed compositions can be administered any time after an autoimmune disease is diagnosed. In one aspect, the IL-2C composition is administered 1, 2, 3, 4, 5 6, 7, 8, 9, 10, 11, 12, 13, 14, 15, 16, 17, 18, 19, 20, 21, 22, 23, 24, 25, 26, 27, 28, 29, 30, 31, 45, 58, 59, 60, 61, 75, 90 days, 4, 5, 6, 7, 8, 9, 10, 11, 12, 18 months, 2, 3, 4, 5, 6, 7, 8, 9, or 10 year after onset of the autoimmune disease.

### E. Methods of Treating Autoinflammatory Disease

The therapeutic benefit of the disclosed compositions is not limited in treatment of inflammation resulting from adaptive immune responses, but are also effective in arresting inflammation-driven destruction associated with the inborn errors of innate immune responses (i.e. Constitutive inflammation that underlies autoinflammatory diseases). As used herein “autoinflammatory diseases refer to disorders where the innate immune response attacks host cells. Examples of autoinflammatory disorders include, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF) and other cryopyrin-associated periodic syndromes (CAPS), Tumor Necrosis Factor (TNF)—Associated Periodic Syndrome (TRAPS), TNFRSF11 A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behgets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome and other Type 1 Interferonopathies, Acute Febrile Neutrophilic Dermatosis, X-linked familial hemophagocytic lymphohistiocytosis, Lyn kinase-associated Autoinflammatory Disease (LAID), and intestinal and skin inflammatory disorders caused by deletion mutation of the carboxy-terminal segment of the NF-κB essential modulator (NEMO). In one aspect, disclosed herein are methods of treating, decreasing, inhibiting, reducing, ameliorating, and/or preventing an autoinflammatory disorder or inflammatory symptoms associated with an autoinflammatory disorder comprising administering to a subject with an autoinflammatory disease a therapeutically effective amount of any of the IL-2C compositions disclosed herein (such as for example IL-2/F5111.2 antibody complex). For example, disclosed herein are methods of treating, decreasing, inhibiting, reducing, ameliorating, and/or preventing an autoinflammatory disease or condition or methods of treating, decreasing, inhibiting, reducing, ameliorating, and/or preventing inflammation caused by an autoinflammatory disease in a subject comprising administering to the subject an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

The disclosed compositions can be administered any time after an autoinflammatory condition or disease is diagnosed. In one aspect, the IL-2C composition is administered 1, 2, 3, 4, 5 6, 7, 8, 9, 10, 11, 12, 13, 14, 15, 16, 17, 18, 19, 20, 21, 22, 23, 24, 25, 26, 27, 28, 29, 30, 31, 45, 58, 59, 60, 61, 75, 90 days, 4, 5, 6, 7, 8, 9, 10, 11, 12, 18 months, 2, 3, 4, 5, 6, 7, 8, 9, or 10 year after onset of the autoinflammatory disease.

### F. Method of Treating Cancer

Inflammation is also a significant part of the pathology resulting from a cancer. Accordingly, in one aspect, disclosed herein are methods of treating, decreasing, inhibiting, reducing, ameliorating, and/or preventing a cancer in a subject or methods of treating, decreasing, inhibiting, reducing, ameliorating, and/or preventing an inflammatory condition or inflammation caused by a cancer comprising administering to the subject any of the IL-2C compositions disclosed herein. For example, disclosed herein are methods of treating, decreasing, inhibiting, reducing, ameliorating, and/or preventing a cancer in a subject or methods of treating, decreasing, inhibiting, reducing, ameliorating, and/or preventing an inflammatory condition or inflammation caused by a cancer comprising administering to the subject an IL-2:anti-IL-2 antibody (Ab) complex (IL-2C), wherein the anti-IL-2 antibody (such as for example the human anti-IL-2 antibody clone F5111.2 or its mouse equivalent JES6-1A12) binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

It is understood and herein contemplated that the disclosed IL-2C compositions (such as for example IL-2/F5111.2 antibody complex) can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

The disclosed compositions can be administered any time after cancerous tissue is detected. In one aspect, the IL-2C composition is administered 1, 2, 3, 4, 5 6, 7, 8, 9, 10, 11, 12, 13, 14, 15, 16, 17, 18, 19, 20, 21, 22, 23, 24, 25, 26, 27, 28, 29, 30, 31, 45, 58, 59, 60, 61, 75, 90 days, 4, 5, 6, 7, 8, 9, 10, 11, 12, 18 months, 2, 3, 4, 5, 6, 7, 8, 9, or 10 year after onset of the cancer.

In one aspect, it is understood the treatment of cancer does not need to be limited to the administration of modified compositions comprising IL-2C (such as, for example an IL-2/F511.2 complex), but can include the further administration of anti-cancer agents to treat, inhibit, reduce, decrease, ameliorate, and/or prevent a cancer or metastasis. Anti-cancer therapeutic agents (such as checkpoint inhibitors, chemotherapeutics, immunotoxins, peptides, and antibodies) that can be used in the methods of treating, inhibiting, reducing, decreasing, ameliorating, and/or preventing a cancer and/or metastasis and in combination with any of the disclosed IL-2C compositions can comprise any anti-cancer therapeutic agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Anti-cancer agents and immune regulators can also include checkpoint inhibitors. Checkpoint inhibitors include, but are not limited to, antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### G. Methods of Enhancing Immune Responses and Establishment of Immunological Memory

Finally, we asked if JES6 IL-2C could be used to deliver physiological IL-2 signals that are required for memory establishment to conventional CD25-expressing anti-viral CD4 T effector cells responding to infection. We thus tested if JES6 IL-2C could rescue memory formation by IL-2-deficient (Il2^{-/-}) CD4 T cells responding to IAV that fail to survive long-term without receipt of IL-2 signals during 5-7 day post-infection (dpi). JES6 IL-2C rescued Il2^{-/-} CD4 T cell memory formation to a similar degree as that observed with S4B6 IL-2C. The results thus demonstrate that CD25-targeted IL-2C can deliver physiologically relevant IL-2 signals that promote anti-viral memory CD4 T cell formation while simultaneously promoting tissue integrity during pathogen challenge.

Accordingly, disclosed herein are methods of enhancing or increasing an immune response (including but not limited to responses from T cells (such as, for example, and increase in CD8 T cells (including, but not limited to, effector, central memory, effector memory, and peripheral memory CD8 T cells), CD4 T cells (including, but not limited to T_{H}1, T_{H}2, and T_{H}17 CD4 T cells), B cell, regulatory CD4 T cells (Tregs), NK cells, NK T cells, γδ T cells, innate lymphoid cells (including, but not limited to ILC1, ILC2, and ILC3)) and/or enhancing or increasing the formation of immunological memory (including, but not limited to memory CD8 T cells (including, but not limited to, central memory, effector memory, and peripheral memory CD8 T cells), memory CD4 T cells (including, but not limited to T_{H}1, T_{H}2, and T_{H}17 CD4 T cells), memory B cells, plasma cells, memory NK cells, and memory NK T cells), to a microbial infection, autoimmune disease, autoinflammatory disease, or cancer in a subject comprising administering to the subject any of the IL-2C compositions disclosed herein. For example, disclosed herein are methods of enhancing or increasing a T cell response to a microbial infection, autoimmune disease, autoinflammatory disease, or cancer in a subject comprising administering to the subject an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

The disclosed compositions can be administered to enhance immune responses or the establishment of immunological memory any time during prior to the establishment of immunological memory and thus can be administered 1, 2, 3, 4, 5 6, 7, 8, 9, 10, 11, 12, 13, 14, 15 days after the onset of the microbial infection and/or cancer.

### H. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: CD 25-Targeted IL-2 Signals Promote Improved Outcomes of Influenza Infection and Boost Memory CD4 Cell Formation

a) Results

(1) JES6 IL-2C Induce Systemic Inflammation when Delivered to Unprimed Mice

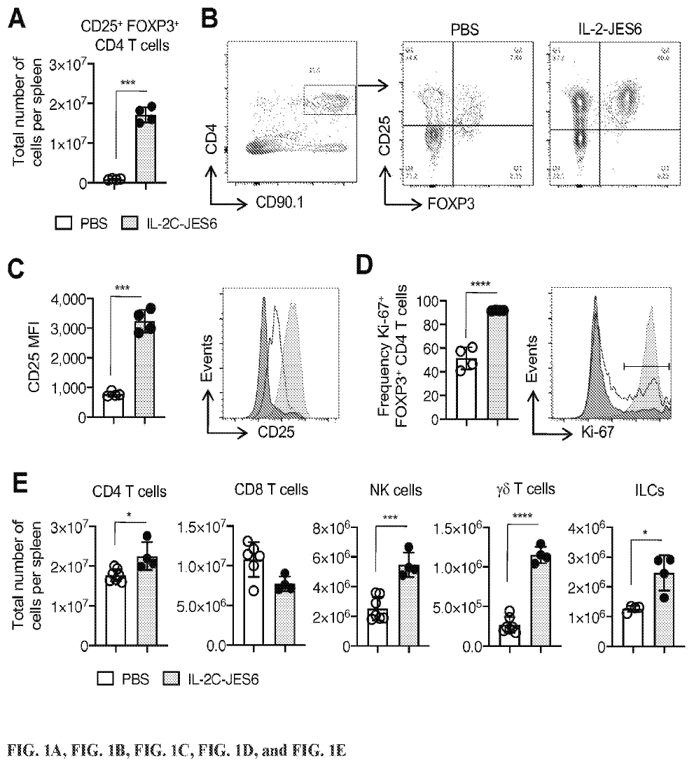

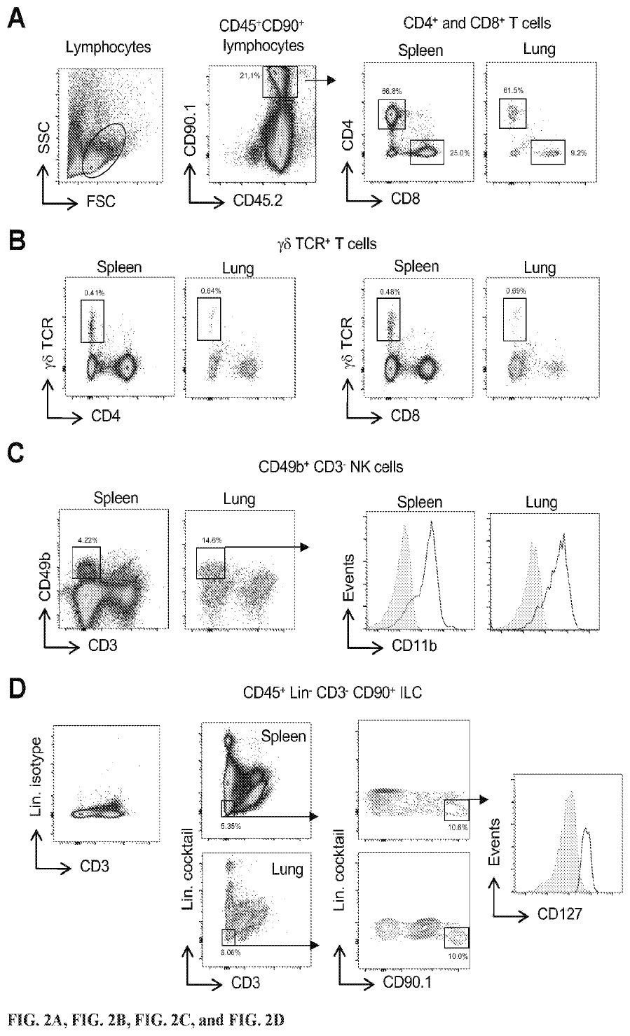

We first delivered JES6 IL-2C containing 2 μg of recombinant murine IL-2 to naive mice by intraperitoneal (i.p.) injection for 3 consecutive days. This is the same treatment regime we used to test the impact of S4B6 IL-2C during IAV infection. The mice were analyzed on the fourth day after initiation of treatment and were compared to control mice receiving PBS. First, we confirmed the expected activity of JES6 IL-2C in dramatically increasing the number of CD25⁺FOXP3⁺CD4 Tregs in the spleen (FIGS. 1a and 1b). JES6 IL-2C treatment significantly increased the mean expression of CD25 on FOXP3⁺CD4 T cells (FIG. 1c). We found the upregulated CD25 expression on FOXP3⁺ T regs in JES6 IL-2C treated mice to associate with roughly a 2-fold increase in the frequency of FOXP3⁺ cells high for the proliferation marker Ki-67 (FIG. 1d). In JES6 IL-2C treated mice, we also found small but significant increases in total CD4 T cells but not CD8 T cells, a 2-fold increase in NK cells, and a 4-fold increase in γδ T cell numbers (FIG. 1e). These results for CD8 T cells and NK cells are not as marked as those observed previously where CD8 T cells and NK cells expand more than 4- and 16-fold, respectively, following S4B6 IL-2C administration. Given published observations of IL-2-dependent expansion of ILC, we also assessed whether ILC were impacted by IL-2C treatment. JES6 IL-2C administration significantly increased ILCs. The gating strategies used are shown in FIG. 2.

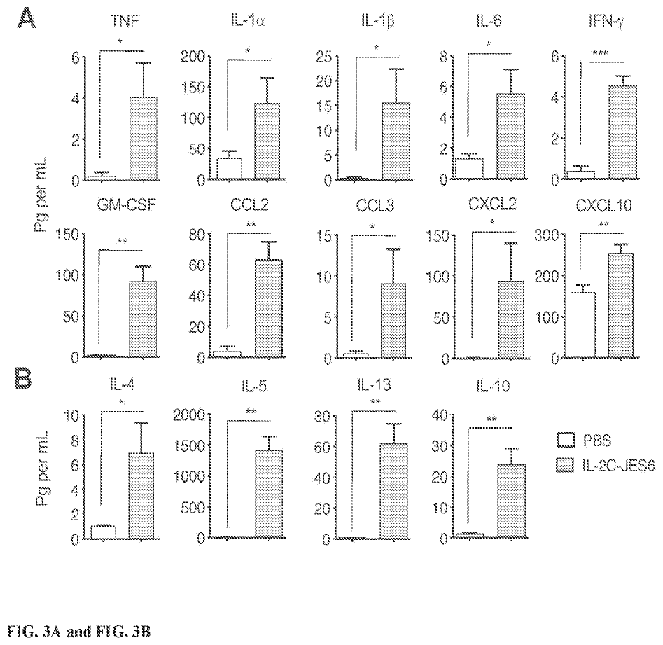

We next assessed protein levels of a broad array of cytokines and chemokines in the serum on the fourth day following JES6 IL-2C treatment and compared levels to those detected in mice receiving PBS alone. Surprisingly, despite its widespread use as an anti-inflammatory agent, JES6 IL-2C treatment significantly increased levels of a number of prototypical pro-inflammatory factors including TNF, IL-1, IL-6, and IFN-γ (FIG. 3a). Treatment also enhanced levels of cytokines typically associated with Th2 and ILC responses including IL-4, IL-5, IL-13, as well as IL-10 (FIG. 3b).

(2) JES6 IL-2C Induce High Levels of Lung Inflammation

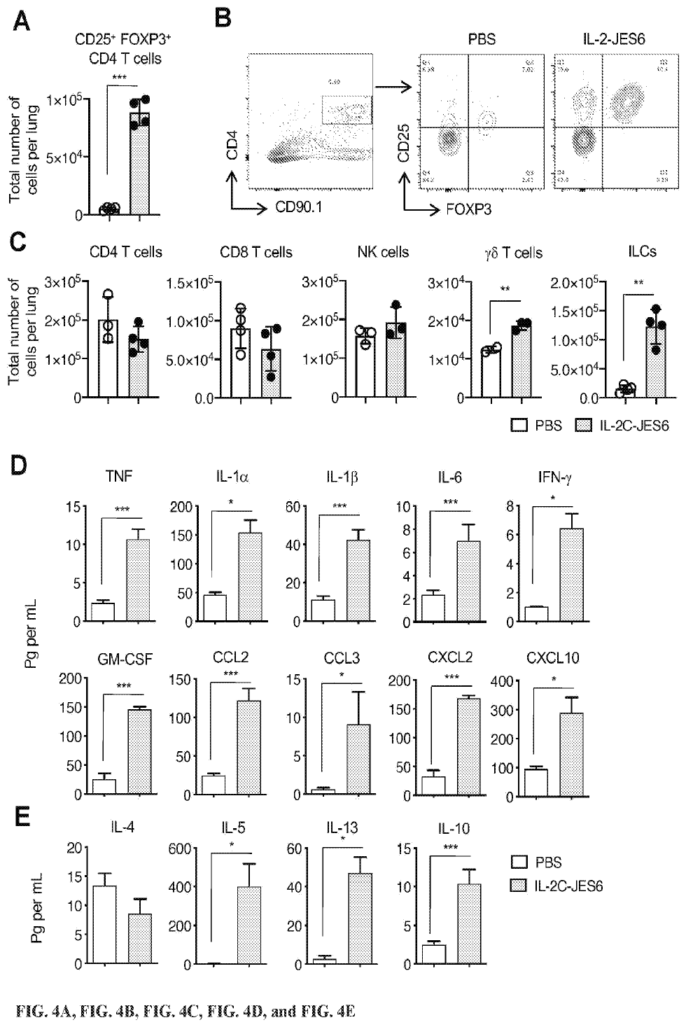

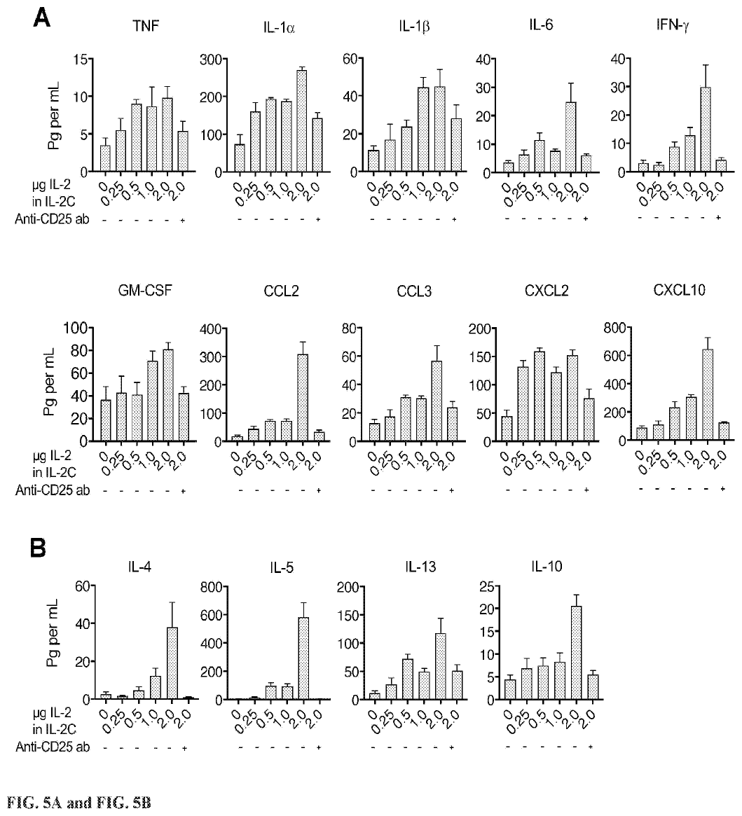

Given that the lung is particularly sensitive to IL-2-driven inflammation, we analyzed the impact of JES6 IL-2C treatment on cellular subsets and inflammatory mediators in the lung. As compared to the spleen, which increases in total cellularity following IL-2C treatment by 2-3-fold, significant increases in total cellularity in the lungs were not observed. However, similar to the expansion of CD25⁺FOXP3⁺CD4 T cells (Tregs) in the spleen following JES6 IL-2C treatment, a robust increase in CD25⁺FOXP3⁺CD4 T cells was observed in the lung (FIGS. 4a and 4b). A small but significant increase in lung γδ T cells and a 3-fold expansion of ILC were also detected, whereas total CD4 and CD8 T cells, and NK cells numbers were not impacted (FIG. 4c). Nevertheless, most of the inflammatory factors that were found to be elevated in the serum in FIG. 1 were also detected at higher levels in the lungs of mice treated with JES6 IL-2C versus in control animals (FIGS. 4d and 4e).

We titrated the amount of recombinant IL-2 delivered during IL-2C treatment and found that while some factors were significantly enhanced when less IL-2 was used, the 2 μg dose was required to see robust, widespread effects in the lung (FIG. 5). To confirm that the IL-2C promote inflammatory responses by binding to CD25⁺ cells we pretreated mice with blocking antibody against CD25 by i.p. injection and the next day administered JES6 IL-2C for 3 consecutive days. Blocking CD25 abrogated the impact of the JES6 IL-2C (FIG. 5), confirming that IL-2C binding to the CD25 receptor is required and ruling out that contaminants in reagents or unexpected binding of IL-2 to the CD122 component of the IL-2 receptor, or other receptors, is responsible for the proinflammatory impacts observed.

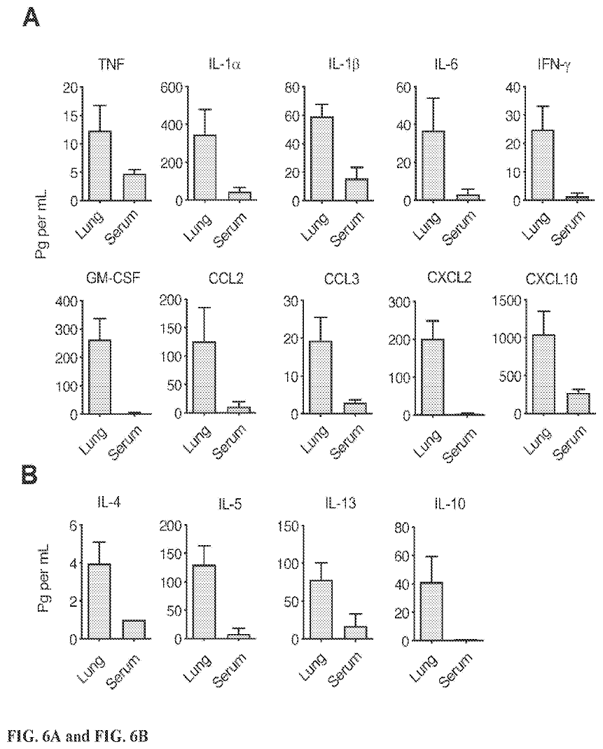

The factors detected in the lungs in FIG. 4 can arise from local impacts of JES6 IL-2C or may have originated from systemic cellular sources. We thus delivered the IL-2C to mice by intranasal (i.n.) administration to analyze the local versus systemic impact. We observed markedly higher levels of inflammation at 4 days post-treatment in the lung versus in the serum following i.n. IL-2C administration, strongly supporting that CD25⁺ cells in the lungs can respond to IL-2 and promote strong local inflammation in the absence of pathogen infection (FIG. 6). These results demonstrate the acute induction of pro-inflammatory mediators in the lung by the JES6 IL-2C and reinforce that the lung environment is particularly sensitive to IL-2-induced inflammatory signals.

(3) JES6 IL-2C Boost Inflammation but Restrain Immunopathology During IAV Infection

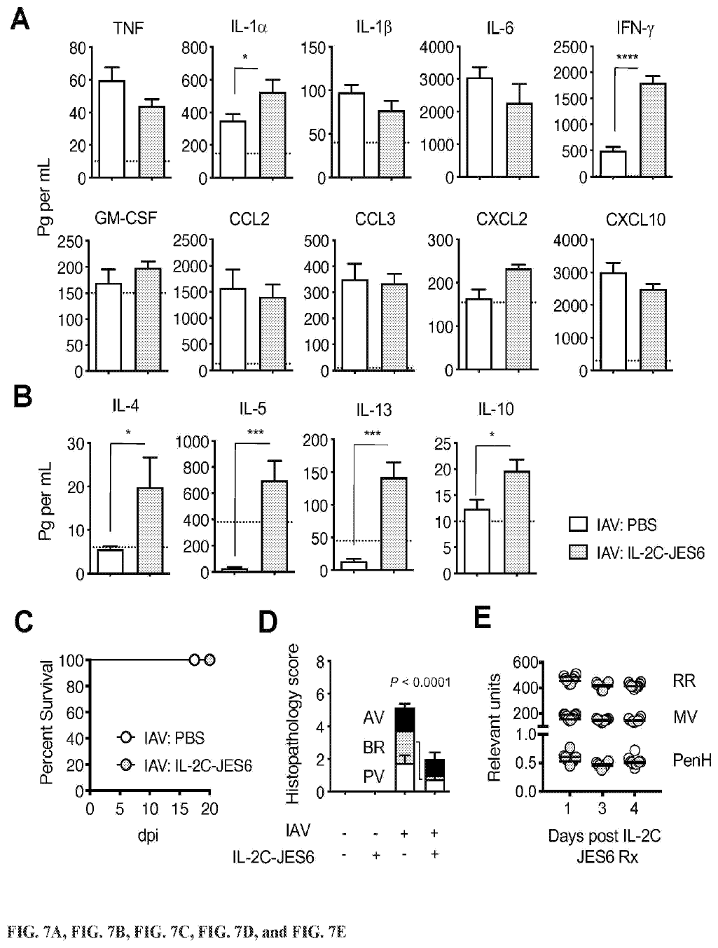

Given the unexpected ability of JES6 IL-2C to concurrently boost T regs and lung inflammation, we infected mice with a low dose of IAV and on the same day, initiated treatment with IL-2C for three days and assessed infection outcomes. Only one inflammatory cytokine, IFN-γ was detected at markedly higher levels in the lungs of IAV infected mice treated with JES6 IL-2C than in mice infected with IAV or treated with JES6-IL-2C alone (FIG. 7a). In contrast, treatment of IAV-infected mice with S4B6 IL-2C i.p. in this model drives heightened lung levels of many inflammatory cytokines and chemokines (IL-6, IFN-γ, IL-17, CCL2, CCL3, and G-CSF) beyond those seen in mice treated with IL-2C alone or in mice only infected with IAV. We also observed significant but more restrained increases in the levels of IL-1α, IL-4, IL-5, IL-13, and IL-10 in the lungs of IAV infected mice treated with JES6 IL-2C (FIG. 7b).

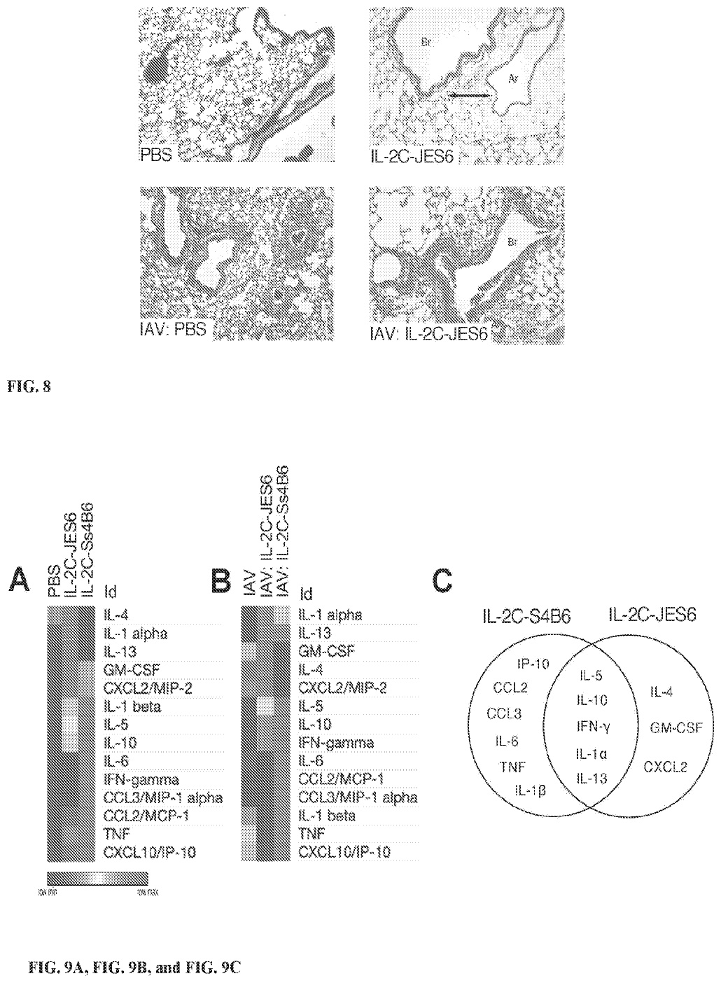

We thus next tested whether IL-2 targeted to CD25⁺ cells would impact survival following sublethal IAV challenge. S4B6 IL-2C administration for 4 instead of 3 days resulted in the acute death of all mice challenged with a normally sublethal dose of IAV without altering immunopathology in the lungs. When the same treatment regime was employed, no mice that received JES6 IL-2C and sublethal IAV infection succumbed to infection (FIG. 7c). Remarkably, despite the pro-inflammatory impact of the JES6 IL-2C in terms of induction of soluble factors, there was a striking decrease in the extent of histological changes observed in the lungs of mice treated with JES6 IL-2C and IAV versus mice infected with IAV alone. This was most apparent in terms of reduced bronchial inflammation, which was virtually absent in IAV-challenged mice treated with JES6 IL-2C versus in mice infected with IAV alone (FIGS. 7d and 8). These results reveal a surprising disconnect in terms of levels of soluble factors in the lung typically associated with damaging inflammation and the degree of immunopathology observed during acute pulmonary viral infection.

Even in the absence of IAV infection, S4B6 IL-2C-induced inflammation correlated with acute impairment of respiratory mechanics. We therefore tested the extent to which JES6 IL-2C treatment in the absence of IAV infection impacted lung function of mice. Despite the induction of inflammation summarized in FIG. 3, JES6 IL-2C did not affect breathing as measured by several parameters (FIG. 7e), which we found to be significantly altered by S4B6 IL-2C administration.

(4) Distinct and Overlapping Patterns in JES6 Versus S4B6 IL-2C Induced Inflammation

The results here demonstrate that S4B6 and JES6 IL-2C both enhance the production of soluble mediators of inflammation, but have dramatically different impacts on respiratory functions and immunopathology. We thus asked if we could identify patterns within the inflammatory responses induced by treatment with these IL-2C. We first compared the relative impact of S4B6 and JES6 IL-2C treatment on cytokines and chemokines detected in the lung in the absence of infection within the same experiment. We present heatmaps based on average protein expression to better visualize and compare the scope of the two distinct inflammatory responses. FIG. 9a summarizes the expression patterns of analytes significantly upregulated in the lung by either JES6 or S4B6 IL-2C treatment. A complex pattern of overlapping and unique induction of distinct factors is evident. Both complexes induce the upregulation of the majority of mediators assessed and in general, the upregulation of analytes impacted by both treatments was higher with S4B6 versus JES6 IL-2C. In the absence of infection, JES6 IL-2C uniquely induced higher levels of IL-1α, IL-4, and IL-13. These results support that while S4B6 and JES6-based IL-2C have some overlapping impacts on acute inflammation, each IL-2C also induces a distinct suite as well as different levels of proinflammatory factors.

We reasoned that this approach can provide insight into the beneficial versus most deleterious elements of the ‘cytokine storm’ associated with detrimental outcomes of severe IAV infection. We thus compared levels of proinflammatory factors in lung homogenates of groups of mice on day 4 after either infection with low-dose IAV alone, or infection with IAV and treatment for 3 days with either S4B6 or JES6 IL-2C. A heatmap summarizing those inflammatory factors upregulated by either IL-2C treatment versus levels detected in mice only challenged with IAV is shown in FIG. 9b. Improved outcomes associated with reduced immunopathology following treatment with JES6 IL-2C during IAV infection correlate with increased expression of IL-4, GM-CSF, and CXCL2. Conversely, detrimental outcomes in IAV infected and S4B6-treated mice was associated with higher levels of many classic pro-inflammatory factors including TNF, IL-6, CCL2, and CCL3 and IL-1β. Thus, while there is considerable overlap in the inflammatory signatures in IAV-infected mice that are treated with S4B6 and JES6 IL-2C, unique patterns in the response are evident (FIG. 9c).

(5) JES-6 IL-2C Complexes can Signal to CD4 T Cells to Rescue Memory Formation

In addition to driving inflammation and modulating diverse lymphocyte populations, IL-2 is a key signal in promoting T cell memory. IL-2-dependent signals required for IAV-specific CD4 T cells responding to IAV to form memory can be delivered to Il2^{-/-} CD4 T cells by treating mice with S4B6 IL-2C from 5-7 dpi. IL-2 signals promote upregulation of the IL-7 receptor (CD127) on the surface of effector CD4 T cells at 7 dpi, thus increasing their memory fitness versus cells expressing less IL-7 receptor. Given that JES6 IL-2C reduce immunopathology associated with IAV infection while S4B6 IL-2C instead promote immunopathology, we asked if JES6 IL-2C could be employed to rescue memory formation from Il2^{-/-} CD4 T cells responding to IAV. We thus treated recipients of Il2^{-/-} DO11.10 cells that were challenged with PR8-OVA_{II} with JES6 or S4B6 IL-2C between 5-7 dpi and assessed donor cell recovery at 7 dpi, the peak of expansion, and at 28 dpi, a memory timepoint.

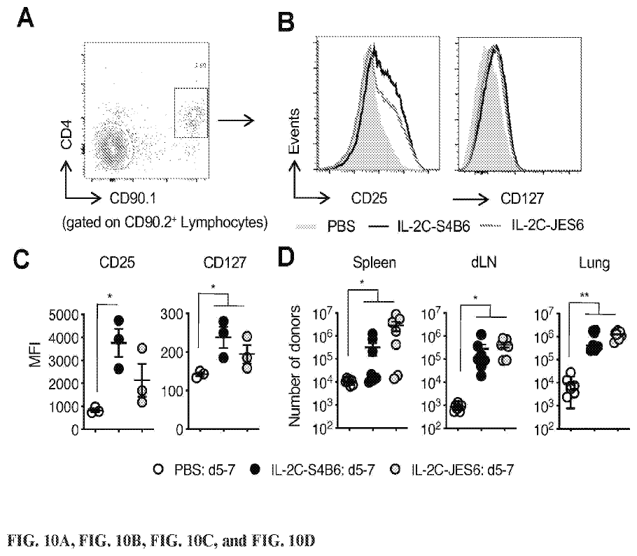

S4B6 and JES6 IL-2C both increased CD25 expression on the surface of 112-donor CD4 T cells at the effector phase of the response, though the upregulation associated with JES6 IL-2C was not significantly enhanced compared to untreated mice, perhaps due to binding of the JES6 IL-2C to CD25 (FIGS. 10a and 10b). Nevertheless, S4B6 and JES6 IL-2C similarly upregulated CD127 expression versus expression on donor cells in mice receiving PBS alone (FIGS. 10c and 10b). At 28 dpi, we observed near identical rescue of memory formation from the Il2^{-/-} donor cells in the spleen, dLN, and especially the lung, where the number of donor cells was about 2 logs increased in mice receiving either IL-2C versus in mice not receiving IL-2C (FIG. 10c). These results indicate that JES6 IL-2C delivered systemically can efficiently deliver pro-memory IL-2 signals to CD4 T cells responding in vivo.

b) Discussion

Although using IL-2 to promote or inhibit immune responses in clinical settings is gaining momentum, how IL-2 acts to shape complex inflammatory responses is still not well-understood. Furthermore, given that IL-2 available for paracrine consumption during immune responses can signal cells expressing high CD122 and/or high CD25, an analysis of how CD25-targeted IL-2 impacts inflammatory responses is required. This is also an important consideration for the myriad of experimental models that use IL-2C administration as a tool for the targeted expansion of specific subsets of lymphocytes, as this strategy can also have off target effects that impact the results observed. Indeed, IL-2 produced by CD4 T cells responding to IAV, or S4B6 IL-2C given to IAV challenged mice, markedly enhances a broad spectrum of inflammatory cytokines and chemokines both systemically and in the infected lung. This IL-2-induced inflammatory response correlated with reduced lung function, less efficient viral clearance, and enhanced weight loss. We show here using the same experimental system that administration of JES6-based IL-2C also drives a strong, acute inflammatory response under steady-state conditions and during viral infection. However, in contrast to results observed with S4B6 IL-2C, JES6 IL-2 administration correlates with improved outcomes after IAV infection.

We provide several novel findings demonstrating that CD25-targeted IL-2 complexes delivered systemically induce a broad range of inflammatory factors systemically and in the lung in otherwise naive mice. An even stronger local response in the lung is generated upon intranasal JES6 IL-2C administration. These observations are surprising for two reasons. The first being that expression of CD25 is most-often tied to lymphocytes in activated states, most notably on T cells in which high CD25 levels are maintained only short-term following antigen stimulation, and relatively few highly activated cells are expected to populate the steady state. The second is the well-known ability of JES6 IL-2C to selectively promote FOXP3⁺ Treg expansion, as this subset constitutively expresses CD25, an outcome commonly associated with anti-inflammatory impacts.

JES6 IL-2C have recently been used to expand innate lymphoid cells in vivo. We speculate that ILC contribute to the ‘Th2’-associated cytokines (IL-4, IL-5, and IL-13) induced by JES6 IL-2C administration. In addition, as ILC have been implicated as key players in lung repair following IAV challenge, the activation of ILC by JES6 IL-2C can contribute to the reduced immunopathology seen in the studies. The results as well as other reports indicate that FOXP3⁺ T reg cells have a minimal impact on outcomes of primary IAV infection and play a greater role during secondary infection, where stronger T cell responses in the lung have a greater capacity to cause immunopathology. Interestingly, in addition to CD4 T cell-derived IL-2, ILC3-derived IL-2 has recently been shown to promote T reg homeostasis in the small intestine through an inflammatory axis dependent upon the production of the inflammatory cytokine IL-1. Lymphoid tissue inducer-like ILC1s and lung ILC3s are also capable of producing IL-2 and whether they similarly promote T reg homeostasis remains to be determined. JES6 IL-2C administration also induces a marked expansion of γδ T cells in the spleen and a significant but smaller response in the lung. In contrast to the beneficial outcomes reported here, IL-2 stimulation of γδ T cells and the subsequent production of IL-1 has recently been reported to compromise lung integrity.

The results highlight an unexpected disconnect between the detection of inflammatory factors at a site of infection and the degree of histological changes observed. This finding prompted us to probe whether patterns could be identified in the inflammatory milieu induced by S4B6 IL-2C that correlate with worsened outcomes, and with JES6 IL-2C that correlate with improved outcomes. Indeed, we found striking patterns of inflammation driven by IL-2 that correlated with improved outcomes (higher IL-4, GM-CSF, and CXCL2) or acute death (IL-1p, IL-6, TNF, CCL2, CCL3, and CXCL10). The analysis provides a roadmap to begin to determine positive versus negative elements of the ‘cytokine storm’ associated with severe IAV infection, and thus an approach to develop novel therapeutic interventions to improve clinical outcomes.

Finally, we show that JES6 IL-2C that target CD25 can be used to deliver pro-memory IL-2 signals to CD4 T cells responding to infection in vivo. This may at first glance be surprising given that the CD25 subunit of the IL-2 receptor lacks cytoplasmic signaling capacity. However, given that JES6 IL-2C are well-known to stimulate proliferation of T regs, which is supported here by the observation that the majority of T regs in treated mice are high for the proliferation marker Ki-67, and given that we show that pre-treatment of mice with anti-CD25 antibody abrogates the impacts of JES6 IL-2C, we surmise that the CD25-dependent binding of the IL-2C is able to stimulate similar signaling as S4B6 IL-2C. We stress that induction of different patterns of inflammatory cytokine and chemokine production by JES6 and S4B6 IL-2C but similar rescue of memory formation from Il2^{-/-} CD4 T cells indicates that it is the direct impact of IL-2 signals on the CD4 T cells, and not other aspects of IL-2-induced inflammation that is responsible. This agrees with findings using an adoptive transfer model that show that the inflammatory milieu associated with IAV has a minimal effect in promoting CD4 T cell memory. CD25-targeted IL-2 signals can thus be developed into powerful clinical approaches to simultaneously improve T cell memory while protecting tissue integrity.

In summary, we provide novel data indicating that prototypical anti-inflammatory IL-2C made with recombinant IL-2 and the anti-IL-2 antibody clone JES6-1A12 are capable of driving a robust, acute systemic inflammatory response when administered i.p., and a local inflammatory response in the lungs when delivered i.n. Surprisingly, this inflammatory response improves rather than worsens immunopathology in the lung during IAV infection. Comparing the patterns of cytokine and chemokine upregulation by JES6- and S4B6-based IL-2C demonstrates remarkably stable hallmarks in both the steady state and during infection. The analysis provides an avenue for determining the most detrimental elements of the ‘cytokine storm’ induced by influenza versus those aspects that may help to counter tissue-damage, or that correlate with this activity. The studies also indicate that properly timed IL-2 signals can be used to simultaneously protect against tissue damage and promote robust CD4 T cell memory during IAV infection.

c) Methods

(1) Mice

BALB/c Thy1.2 or BALB/c Thy1.1 mice were used in experiments when 8 to 12 weeks old. Naive CD4 T cells were obtained from 5 to 8-week old male or female Il2^{-/-} DO11.10 Thy1.2 or Thy1.2/Thy1.1 mice originally provided by A. Abbas (UCSF). BALB/c and DO11.10 mice were bred in the vivarium of the Trudeau Institute, the University of Massachusetts Medical School, or the University of Central Florida.

(2) Cytokine Complex and Receptor Blockade Treatments

Mice were treated for the indicated days with IL-2 complexes (IL-2C) that consisted of 2 μg per day of recombinant IL-2 (ThermoFisher) pre-mixed with 20 μg of anti-mouse IL-2 monoclonal antibody clone JES6-1A12 (JES6) (ThermoFisher), or IL-2C pre-mixed with IL-2 and the anti-IL-2 antibody clone S4B6 (BD Biosciences). In certain experiments, the amount of IL-2 in the complexes was varied, as indicated. Complexes were incubated at room temperature for 20 minutes (min.) before intraperitoneal (i.p.) injection in 200 μL of PBS. Control mice received 200 μL of PBS alone.

For some experiments, mice were treated as indicated with 0.25 mg of anti-CD25 (IL-2 Rα) antibody (clone PC-61.5.3, BioXcell) to block IL-2 signaling one day prior to initiation of IL-2C treatment. Antibody was delivered by i.p. injection in 200 μL of PBS.

(3) Virus stocks and infections

Influenza A/Puerto Rico/8/1934 (PR8) (H1N1) originating from stocks prepared at the Trudeau Institute and in use in experiments since 1997, and A/PR8-OVA_{II} (H1N1) from stock obtained from P. Doherty at St Jude's Children's Hospital, were produced in the allantoic cavity of embryonated hen eggs at the Trudeau Institute and the lethal dose (LD₅₀), egg infective dose (EID₅₀) or tissue culture infective dose (TCID₅₀) characterized. Mice were infected intranasally under light isoflurane anesthesia (Webster Veterinary Supply) with a sublethal 0.2 LD₅₀ dose of virus in 50 μl PBS and morbidity and mortality monitored.

(4) Tissue Preparation and Flow Cytometry

At different time points after IL-2C treatment and or virus infection, blood and lungs were obtained from euthanized animals for Luminex multiplex analysis. Lungs were harvested and homogenized in RPMI 1640 media supplemented with 2 mM L-glutamine, 100 IU penicillin, 100 μg per mL streptomycin (Invitrogen), 10 mM HEPES (Research Organics), 50 μM 2-mercaptoethanol (Sigma-Aldrich) and 7.5% fetal bovine serum (Hyclone) and serum collected from blood.

Alternatively, for flow cytometry, mice were euthanized by cervical dislocation followed by exsanguination by perforation of the abdominal aorta. Lungs were perfused by injecting 10 ml of PBS in the left ventricle of the heart. Lungs and spleen were prepared into single cell suspensions by mechanical disruption of organs and passage through a nylon membrane. Flow cytometry was performed using fluorochrome-labeled antibodies at manufacturer's recommended dilutions for surface staining including anti-Thy1.1 (OX-7), anti-Thy1.2 (53-2.1), anti-CD4 (RM4.5 and GK1.5), anti-CD8 (53-6.7), anti-CD45.2 (104), anti-γδ TcR (GL3), anti-CD3 (17A2), anti-CD25 (PC61), anti-CD11b (M1/70), anti-Gr-1 (RB6-8C5), anti-CD127 (A7R34), anti-CD49b (DX5), and murine hematopoietic lineage antibody cocktail containing anti-CD3 (17A2), anti-CD45R/B220 (RA3-6B2), anti-CD11b (M1/70), anti-TER-119 (TER-119), anti-Ly-G6/Gr-1 (RB6-8C5). Innate lymphoid cells (ILCs) were identified as CD45⁺, Lineage⁻, CD3⁻, CD90⁺, CD127⁺ (IL-7R) lymphocytes.

Intracellular staining for FOXP3 and K1-67 was performed as per manufacturer's instructions with the FOXP3 Transcription Factor Fixation/Permeabilization Concentrate and Diluent (Life, eBioscience) and fluorochrome-labeled anti-FOXP3 (FJK-16s) and K1-67 (SolA15) antibodies. Analysis was performed using FACS Canto II and LSRII instruments (BD Biosciences) and FlowJo (Tree Star) analysis software.

(a) Detection of Inflammatory Cytokines and Chemokines

Levels of cytokines and chemokines in lung homogenates or serum were determined using mouse multiplex kits (Invitrogen and Millipore) read on a Bio-Plex Multiplex 200 Luminex reader (Bio-Rad) as per manufactures' instructions. The assay sensitivity for 12 of the 14 the analytes presented is below 1 μg/mL, ranging from 0.03 μg/mL to 0.69 μg/mL, and is 1.16 μg/mL and 3.43 μg/mL for the remaining analytes CXCL2 and CCL2, respectively.

(b) Histology

For assessment of immunopathology following viral infection and IL-2C treatment, lungs lobes were isolated and immediately fixed in 10% neutral buffered formalin. Lung samples were subsequently processed, embedded in paraffin, sectioned, placed on L-lysine-coated slides, and stained with Hematoxylin and Eosin (H&E) using standard histological techniques at the Morphology Core at UMMS. Triplicate non-serial sections were graded blindly from 0 to 4, for the extent of inflammatory cell infiltration and damage of bronchi, arteries or alveoli by a certified pathologist.

(c) Measurement of Pulmonary Mechanics

Non-invasive whole-body plethysmography (WBP) (Buxco) was employed to measure respiratory rates (breaths per min.), minute volumes (mL per min.), and enhanced pause PenH, on conscious, unrestrained animals following IL-2C treatment. The minute volume is defined as the volume of air exchanged during a 1-min. interval and is calculated as follows [respiratory rate X tidal volume].

(5) CD4 T Cell Isolation and In Vitro-Primed Memory Generation

Naive CD4⁺ T cells were obtained from pooled spleen and peripheral lymph nodes. Briefly, cells were purified by nylon wool and percoll density gradient separation. CD4 T cells were isolated by positive CD4 MACS selection (Miltenyi). Resulting CD4 cells routinely expressed a characteristic naive phenotype (small size, CD62L^{hi}, CD44^{lo} and CD25^{lo}) >97% TcR+. T_{H}1-polarized effectors were generated in vitro as described. Briefly, naive I12-CD4 T cells were cultured with an equal number of irradiated APC (2×10⁵ per mL) in the presence of exogenous IL-2 (20 ng per mL), 2 ng per mL IL-12 (Peprotech), 10 μg per mL anti-IL-4 antibody (1I B11; Bioxcell), and 5 μM OVA_{II} peptide. In vitro-primed memory cells were obtained by thoroughly washing effector cultures at 4 days and re-culturing the cells in fresh media for at least 3 days in the absence of Ag and exogenous cytokines. Live cells were isolated by Lympholyte separation (Cedarlane). All donor CD4 T cells were adoptively transferred in 200 μl phosphate buffered saline (PBS) by intravenous (i.v.) injection. A number of donor cells previously determined to be detectable at the memory phase, 2×10⁶, was transferred. Donor cell injection and viral infection occurred on the same day.

(6) Statistical Analysis

Group sizes of n=3 to 6 were employed for all experiments. For Unpaired, Students t-tests, ∝=0.05, were used to assess whether the means of two normally distributed groups differed significantly. One-way ANOVA analysis with the appropriate multiple comparison post-test, Bonferroni's or Tukey's, was employed to compare multiple means. All error bars represent the standard deviation. Significance is indicated as *P<0.05, **P<0.005, ***P<0.001, ****P<0.0001.

### L. References

Banchereau, J., V. Pascual, and A. O'Garra. 2012. From IL-2 to IL-37: the expanding spectrum of anti-inflammatory cytokines. Nat Immunol 13: 925-931.
Bautista, B. L., P. Devarajan, K. K. McKinstry, T. M. Strutt, A. M. Vong, M. C. Jones, Y. Kuang, D. Mott, and S. L. Swain. 2016. Short-Lived Antigen Recognition but Not Viral Infection at a Defined Checkpoint Programs Effector CD4 T Cells To Become Protective Memory. J Immunol 197: 3936-3949.
Boyman, O., and J. Sprent. 2012. The role of interleukin-2 during homeostasis and activation of the immune system. Nat Rev Immunol 12: 180-190.
Boyman, O., M. Kovar, M. P. Rubinstein, C. D. Surh, and J. Sprent. 2006. Selective stimulation of T cell subsets with antibody-cytokine immune complexes. Science 311: 1924-1927.
Brincks, E. L., A. D. Roberts, T. Cookenham, S. Sell, J. E. Kohlmeier, M. A. Blackman, and D. L. Woodland. 2013. Antigen-specific memory regulatory CD4+Foxp3+ T cells control memory responses to influenza virus infection. J Immunol 190: 3438-3446.
Califano, D., Y. Furuya, S. Roberts, D. Avram, A. N. J. McKenzie, and D. W. Metzger. 2018. IFN-gamma increases susceptibility to influenza A infection through suppression of group II innate lymphoid cells. Mucosal Immunol 11: 209-219.
Cao, Q., R. Wang, Y. Wang, Z. Niu, T. Chen, C. Wang, L. Jin, Q. Huang, Q. Li, X. M. Wang, F. Azmi, V. W. S. Lee, Y. M. Wang, G. Zheng, S. I. Alexander, and D. C. H. Harris. 2020. Regulatory innate lymphoid cells suppress innate immunity and reduce renal ischemia/reperfusion injury. Kidney Int 97: 130-142.
Crellin, N. K., S. Trifari, C. D. Kaplan, N. Satoh-Takayama, J. P. Di Santo, and H. Spits. 2010. Regulation of cytokine secretion in human CD127(+) LTi-like innate lymphoid cells by Toll-like receptor 2. Immunity 33: 752-764.
Dhume, K., and K. K. McKinstry. 2018. Early programming and late-acting checkpoints governing the development of CD4 T-cell memory. Immunology 155: 53-62.
Dooms, H., K. Wolslegel, P. Lin, and A. K. Abbas. 2007. Interleukin-2 enhances CD4+ T cell memory by promoting the generation of IL-7Ralpha-expressing cells. J Exp Med 204: 547-557.
Gu, Y., A. C. Hsu, Z. Pang, H. Pan, X. Zuo, G. Wang, J. Zheng, and F. Wang. 2019. Role of the Innate Cytokine Storm Induced by the Influenza A Virus. Viral Immunol 32: 244-251.
Kamimura, D., and M. J. Bevan. 2007. Naive CD8+ T cells differentiate into protective memory-like cells after IL-2 anti IL-2 complex treatment in vivo. J Exp Med 204: 1803-1812.
Kondrack, R. M., J. Harbertson, J. T. Tan, M. E. McBreen, C. D. Surh, and L. M. Bradley. 2003. Interleukin 7 regulates the survival and generation of memory CD4 cells. J Exp Med 198: 1797-1806.
Krieg, C., S. Letourneau, G. Pantaleo, and O. Boyman. 2010. Improved IL-2 immunotherapy by selective stimulation of IL-2 receptors on lymphocytes and endothelial cells. Proc Natl Acad Sci USA 107: 11906-11911.
Li, J., G. Huston, and S. L. Swain. 2003. IL-7 promotes the transition of CD4 effectors to persistent memory cells. J Exp Med 198: 1807-1815.
Malek, T. R. 2008. The biology of interleukin-2. Annu Rev Immunol 26: 453-479.
Malek, T. R., and A. L. Bayer. 2004. Tolerance, not immunity, crucially depends on IL-2. Nat Rev Immunol 4: 665-674.
McKinstry, K. K., F. Alam, V. Flores-Malavet, M. Z. Nagy, S. Sell, A. M. Cooper, S. L. Swain, and T. M. Strutt. 2019. Memory CD4 T cell-derived IL-2 synergizes with viral infection to exacerbate lung inflammation. PLoS Pathog 15: e1007989.
McKinstry, K. K., S. Golech, W. H. Lee, G. Huston, N. P. Weng, and S. L. Swain. 2007. Rapid default transition of CD4 T cell effectors to functional memory cells. J Exp Med 204: 2199-2211.
McKinstry, K. K., T. M. Strutt, B. Bautista, W. Zhang, Y. Kuang, A. M. Cooper, and S. L. Swain. 2014. Effector CD4 T-cell transition to memory requires late cognate interactions that induce autocrine IL-2. Nat Commun 5: 5377.
Menoret, A., J. A. Buturla, M. M. Xu, J. Svedova, S. Kumar, V. A. K. Rathinam, and A. T. Vella. T cell-directed IL-17 production by lung granular gammadelta T cells is coordinated by a novel IL-2 and IL-1beta circuit. Mucosal Immunol 11: 1398-1407.
Monticelli, L. A., G. F. Sonnenberg, M. C. Abt, T. Alenghat, C. G. Ziegler, T. A. Doering, J. M. Angelosanto, B. J. Laidlaw, C. Y. Yang, T. Sathaliyawala, M. Kubota, D. Turner, J. M. Diamond, A. W. Goldrath, D. L. Farber, R. G. Collman, E. J. Wherry, and D. Artis. 2011. Innate lymphoid cells promote lung-tissue homeostasis after infection with influenza virus. Nat Immunol 12: 1045-1054.
Newland, S. A., S. Mohanta, M. Clement, S. Taleb, J. A. Walker, M. Nus, A. P. Sage, C. Yin, D. Hu, L. L. Kitt, A. J. Finigan, H. R. Rodewald, C. J. Binder, A. N. J. McKenzie, A. J. Habenicht, and Z. Mallat. 2017. Type-2 innate lymphoid cells control the development of atherosclerosis in mice. Nat Commun 8: 15781.
Prlic, M., D. Kamimura, and M. J. Bevan. 2007. Rapid generation of a functional NK-cell compartment. Blood 110: 2024-2026.
Roediger, B., R. Kyle, K. H. Yip, N. Sumaria, T. V. Guy, B. S. Kim, A. J. Mitchell, S. S. Tay, R. Jain, E. Forbes-Blom, X. Chen, P. L. Tong, H. A. Bolton, D. Artis, W. E. Paul, B. Fazekas de St Groth, M. A. Grimbaldeston, G. Le Gros, and W. Weninger. 2013. Cutaneous immunosurveillance and regulation of inflammation by group 2 innate lymphoid cells. Nat Immunol 14: 564-573.
Roediger, B., R. Kyle, S. S. Tay, A. J. Mitchell, H. A. Bolton, T. V. Guy, S. Y. Tan, E. Forbes-Blom, P. L. Tong, Y. Koller, E. Shklovskaya, M. Iwashima, K. D. McCoy, G. Le Gros, B. Fazekas de St Groth, and W. Weninger. 2015. IL-2 is a critical regulator of group 2 innate lymphoid cell function during pulmonary inflammation. J Allergy Clin Immunol 136: 1653-1663 e1657.
Roman, E., E. Miller, A. Harmsen, J. Wiley, U. H. Von Andrian, G. Huston, and S. L. Swain. 2002. CD4 effector T cell subsets in the response to influenza: heterogeneity, migration, and function. J Exp Med 196: 957-968.
Seehus, C. R., A. Kadavallore, B. Torre, A. R. Yeckes, Y. Wang, J. Tang, and J. Kaye. 2017. Alternative activation generates IL-10 producing type 2 innate lymphoid cells. Nat Commun 8: 1900.
Sell, S., I. Guest, K. K. McKinstry, T. M. Strutt, J. E. Kohlmeier, E. Brincks, M. Tighe, M. A. Blackman, D. L. Woodland, R. W. Dutton, and S. L. Swain. 2014. Intraepithelial T-cell cytotoxicity, induced bronchus-associated lymphoid tissue, and proliferation of pneumocytes in experimental mouse models of influenza. Viral Immunol 27: 484-496.
Smith, K. A. 1984. Interleukin 2. Annu Rev Immunol 2: 319-333.
Thomas, P. G., S. A. Brown, W. Yue, J. So, R. J. Webby, and P. C. Doherty. 2006. An unexpected antibody response to an engineered influenza virus modifies CD8+ T cell responses. Proc Natl Acad Sci USA 103: 2764-2769.
Tomala, J., and M. Kovar. 2016. IL-2/anti-IL-2 mAb immunocomplexes: A renascence of IL-2 in cancer immunotherapy? Oncoimmunology 5: e1102829.
Zhou, L., C. Chu, F. Teng, N. J. Bessman, J. Goc, E. K. Santosa, G. G. Putzel, H. Kabata, J. R. Kelsen, R. N. Baldassano, M. A. Shah, R. E. Sockolow, E. Vivier, G. Eberl, K. A. Smith, and G. F. Sonnenberg. 2019. Innate lymphoid cells support regulatory T cells in the intestine through interleukin-2. Nature 568: 405-409.

1. A composition comprising an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

2. The composition of claim 1, wherein the antibody comprises the anti-IL-2 antibody clone F5111.2.

3. A method of treating a microbial infection, autoimmune disease, autoinflammatory disease, or cancer in a subject comprising administering to the subject the composition of claim 1.

4. A method of treating a microbial infection, autoimmune disease, autoinflammatory disease, or cancer in a subject comprising administering to the subject an IL-2:anti-IL-2 antibody (Ab) complex (IL-2C), wherein the anti-IL-2 antibody binds to the IL-2 at the R46 residue of IL-2 thereby simultaneously sterically blocking IL-2 from binding to the CD122 subunit of the IL-2 receptor and remaining bioavailable to the CD25 subunit of the IL-2 receptor.

5. The method of treating a microbial infection of claim 3, wherein the microbial infection is a viral infection.

6. The method of treating a microbial infection of claim 5, wherein the viral infection is an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

7. The method of treating a microbial infection of claim 6, wherein the virus is influenza A.

8. The method of treating a microbial infection of claim 3, wherein the microbial infection is caused by a bacterial infection.

9. The method of treating a microbial infection of claim 8, wherein the bacterial infection is an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neisseria meningitidis, Neiserria gonorrhea, Pseudomonas aeruginosa, other Pseudomonas species, Haemophilus influenzae, Haemophilus ducreyi, other Haemophilus species, Clostridium tetani, other Clostridium species, Yersinia enterolitica, and other Yersinia species.

10. The method of treating a microbial infection of claim 3, wherein the microbial infection is caused by a fungal infection.

11. The method of treating a microbial infection of claim 10, wherein the fungal infection is an infection with a fungus selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

12. The method of treating a microbial infection of claim 3, wherein the microbial infection is caused by a parasitic infection.

13. The method of treating a microbial infection of claim 12, wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

14. The method treating a microbial infection of claim 3, further comprising administering to the subject an anti-microbial agent.

15. The method of treating a microbial infection of claim 3, wherein the composition is administered 1, 2, 3, or 4 days post infection.

16. The method of treating cancer of claim 3, wherein the cancer comprises a lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

17. The method treating a cancer of claim 16, further comprising administering to the subject an anti-cancer agent.

18. A method of treating an inflammatory condition or reducing inflammation caused by a microbial infection, autoimmune disease, autoinflammatory disease, or cancer in a subject comprising administering to the subject the composition of claim 1.

19. (canceled)

20. The method of treating an inflammatory condition of claim 18 wherein the inflammatory condition comprises acute inflammation, acute respiratory distress syndrome, subacute inflammation, chronic inflammation, organ-specific inflammation, systemic inflammation, or sepsis.

21. A method of enhancing a T cell response to a microbial infection, autoimmune disease, autoinflammatory disease, or cancer in a subject comprising administering to the subject the composition of claim 1.

22. (canceled)
